(19) United States
(12) Patent Application Publication
(10) Pub. No.: US 2007/0197912 A1
(43) Pub. Date: Aug. 23, 2007

(54) Method for scanning and anaysing a three-dimensional structure

(76) Inventor: Jacques Dory, Estoril (PT)

Correspondence Address: BROWDY AND NEIMARK, P.L.L.C.;624 NINTH STREET, NW, SUITE 300, WASHINGTON, DC, 20001-5303, US

(21) Appl. No.: 10/557,908

(22) PCT Filed: May 13, 2004
(86) PCT No.: PCT/IB04/01890

§ 371 Date: Dec. 19, 2006

(30) Foreign Application Priority Data
May 22, 2003 (FR) 03/06366

(51) Int. Cl. A61B 8/00

(52) U.S. Cl. 600/447

(57) ABSTRACT

Method for scanning and analysing a three-dimensional structure by suitably processing signals representing waves, particularly ultrasonic waves reflected or transmitted by said three-dimensional structure, which processing involves restoring or analysing the three-dimensional structure on the basis of data read out of a field memory. The method comprises calculating, for each structural point, the field memory positions containing the signals detected by the detection elements, corresponding to the waves reflected or transmitted by said point, and data in the field memory is read out by a decoding curve. The method includes detecting the sign of the detected signals, detecting a useful zone of said decoding curve in which the signals at the same sign, and calculating the position and the point being analysed on the basis of an integration of the amplitude of the signals detected in the useful zone of the decoding curve.

The present invention relates to a method for scanning and analyzing a three-dimensional structure by suitable processing signals representing of waves, notably ultrasonic waves reflected or transmitted by said three-dimensional structure.

It notably, but not exclusively applies to the making of devices such as ultrasonic scanners, devices for non-destructive testing of objects, sonars, or even radars.

Conventional devices of this type usually involve emission means which emit an incident wave into the medium to be examined and receiving means optionally using all or a part of the emission means which receive the waves reflected by the obstacles encountered by the incident wave. Means are further provided for processing the signals received by the receiving means and for displaying them in a user utilizable form, for example as an image allowing the position of the obstacles generating reflections of the incident wave to be localized.

The most current method consists in using pulsed waves according to a process consisting of transmitting a pulse of ultrasonic waves in a given direction, detecting the return of the echoes, measuring the time elapsed between emission and reception, and inferring the distance therefrom, taking into account the propagation velocity of the ultrasonic wave, and therefore the position of the obstacle which generated each echo. This process is then repeated in different directions, according to a predetermined sweeping law. It then becomes possible to produce images revealing the obstacles detected by the echoes, the position of which is known.

In order to avoid the drawbacks of devices using a sequential processing mode (line by line scanning), emitting into the three-dimensional structure to be scanned a substantially plane wave was suggested, with a relatively large section generated by a probe consisting of a network comprising a plurality of emitting/receiving elements with small dimensions, preferably less than the ultrasonic wavelength, in order to have a very large radiation pattern, these elements being simultaneously driven in parallel. Upon reception, each emitting/receiving element operates independently and therefore receives separately the waves reflected by the obstacles intercepting the beam of ultrasonic waves which is found in its receiving area. After digitization, the information delivered by these emitting/receiving elements (field of reflected waves) is stored in memories, the reading of which is carried out in the reverse direction to that of writing.

Patent U.S. Pat. No. 4,817,434 describes a device comprising one address generator per receiving element of the probe, which provides the address to be read in a field memory corresponding to the point image to be reconstructed. However, this device only allows a relatively slow image reconstruction rate.

The basis for eliminating this drawback, is the observation by the applicant that in a process such as the one described earlier, each point of the object to be scanned gives rise to a wave which is found stored at addresses of a field memory distributed as a pseudo-hyperbolic curve segment, the characteristics of which depend on the position of the point relatively to the probe and to the radiation pattern of each element (this pseudo-hyperbola theoretically being reduced to two asymptotes for points located against the probe).

A method was developed comprising the following steps:
emitting into said structure an incident wave,
receiving waves reflected or transmitted by the obstacles encountered by the incident wave inside said structure, by a plurality of detection elements independent of each other,
storing, after digitization, signals delivered by the detection elements in a field memory comprising one respective line per detection element, and
reconstructing and/or analyzing the three-dimensional structure from information read in the field memory, wherein for each point of the structure, the positions of the field memory containing the signal detected by the detection elements are calculated, corresponding to the waves reflected or transmitted by this point, these positions being calculated by means of an addressing law, the parameters of which depend on the position of this point relatively to the detection elements, and wherein for each point, the lines of the field memory are read at the respective positions calculated beforehand for this point and stored in addressing memories associated with said lines of the field memory, respectively.

A calculation is then applied to the information read for this point in order to obtain a result representative of the significance of the wave reflected or transmitted by this point, during this calculation, all the lines of the field memory are read in parallel for each point at the positions indicated for this point by the associated addressing memories respectively, the calculation of the results being then applied to all the values read in the field memory, this result being subsequently processed or stored in a specific memory.

With this method described in Patents EP 0825 453 B1 and EP 0 872 742 B1 filed on behalf of the applicant, it is possible to obtain remarkable performances in terms of:
inspection rate and this therefore allows parts with large dimensions to be analyzed,
spatial resolution taking into account the large radiation aperture,
reproducibility as the ultrasonic field is emitted by a plane wave.

Nevertheless, experience shows that in certain applications, constraints limit the performances of the method, in particular the processing rate.

The very high spatial resolution obtained with said method leads to very fine images and if the intention is to retain the fineness of the details, and notably to measure the amplitude of the reflected signals accurately, the frame of analysis should be very closely spaced, and in consequence, the number of points to be calculated is large, which slows down the processing rate excessively.

In other words, the maximum amplitude of the echo may not be detected in the analysis frame if it is located between two analysis steps; this error may be a disadvantage if it proves to be necessary to measure the extent of the obstacle with high accuracy. According to the requested accuracy, this may lead to the use of a smaller analysis step, therefore to a larger number of calculated points, which increases the calculation time and reduces the control rate.

It is found that in the method described in Patents EP 0 825 453 B1 and EP 0 872 742 B1 filed on behalf of the applicant, the analysis step is independent of the number of electronic channels; it is desirable that the analysis step be an integral multiple or sub-multiple of the step between detection elements: thus, in the case of a probe comprising 32 detection elements, spaced apart by 0.8 mm, associated with 32 electronic channels, the analysis step may be equal to 1.6 mm, 3.2 mm, 4.8 mm, 6.4 mm, etc.

The case of probing a metal plate with a thickness of 120 mm with a probe of 64 elements with a step of 1 mm and with a 60 MHz sampling frequency of the detected signal may be considered as an example.

The sound velocity in the material to be probed is 6,000 m/s; the round trip travel time is 40 μs.

Knowing that the analysis step of the detected signal is set to the sampling period and taking into account the number of horizontal lines equivalent to the number of elements of the probe, the number of analyzed points is 40×60×64, i.e. 153,600.

The total processing time is 153,600/60 i.e., 2,560 μs, i.e., a rate of 390 Hz.

It is found that the 3 dB side resolution of the method, at low depth, is of the order of 0.25 mm and that the 1 mm analysis step adopted in said example may cause errors larger than 10 dB on the measured amplitude; this difference is unacceptable in most cases.

It is therefore necessary to reduce the analysis step by a factor 4, the processing rate is located below 100 Hz; this rate may become incompatible with the required inspection rate of parts with large dimensions.

More particularly, the object of the invention is therefore to suppress these drawbacks by a method allowing the processing rate to be increased, without ruining the other performances, by modifying the processing parameters according to the location of the processed area of the field memory and allows the rate and/or the accuracy of this processing to be optimized.

Optimization of the resolution and processing time is based on decoding information read in the field memory; thus the so-called “decoding” curve is the readout curve for values read in the field memory; the positions of the field memory are calculated by means of an addressing law, the parameters of which depending on the position of the point to be analyzed relatively to the detection elements.

The so-called “inscribed” curve corresponds to the signals detected by the detection elements which correspond to waves reflected or transmitted by the point or obstacle to be analyzed.

The present invention is based on the observation that, in a process such as the one described earlier, when the decoding curve does not exactly coincide with the curve inscribed in the field memory, the decoding curve may successively detect positive and negative signals.

Accordingly, it proposes a method for scanning and analyzing a three-dimensional structure, from information read in the field memory by a decoding curve, this method comprising the detection of the sign of the detected signals, the detection of a useful area of said decoding curve wherein the signals are of the same sign and calculation of the position of the analyzed point from integration of the amplitudes of the detected signals in said useful area of said decoding curve.

More specifically, this method may comprise:
detection of positive and negative signals,
detection of the centre of said decoding curve,
storing the sign of the signals at the centre of the decoding curve,
detection of a useful area of the decoding curve wherein the signals are of the same sign,
adding the signals of the same sign in the useful area of the decoding curve,
dividing said sum by the number of analyzed signals of the same sign or by a predetermined constant value corresponding to the total opening of the decoding curve,
storing the result obtained for the analyzed point of the three-dimensional structure.

Optionally, the method according to the invention may implement, from information read in an analysis frame and during the storage phase in the field memory:
the use of a variable horizontal step depending on the depth of analysis, and/or
the change in the horizontal step in the vicinity of the obstacle, and/or
the processing of signals detected prior to the storage phase.

An embodiment of the method according to the invention will be described hereafter, as a non-limiting example, with reference to the associated drawings wherein:

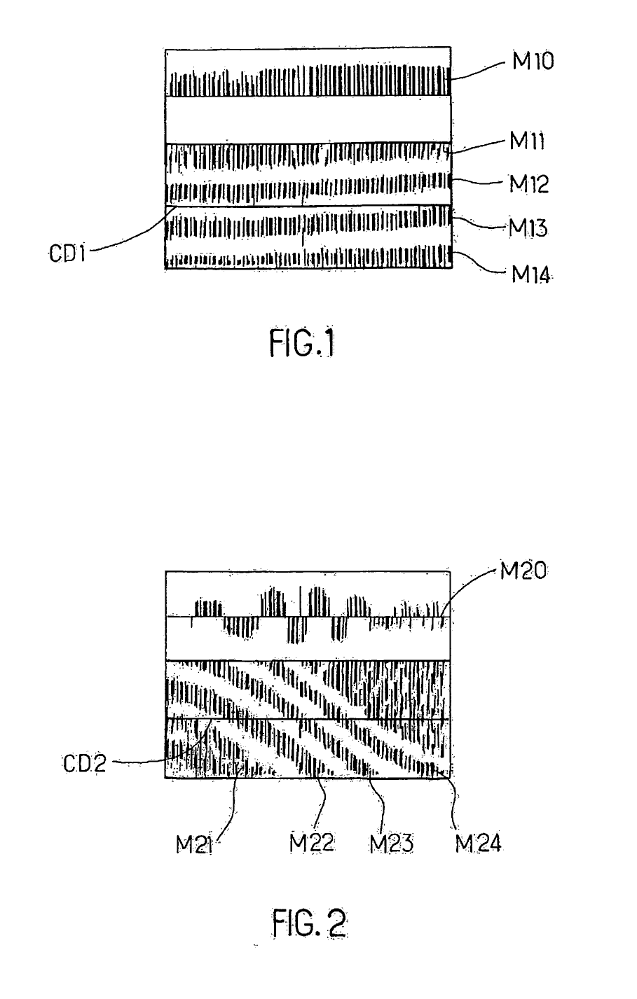

FIG. 1 illustrates the detected signals on the decoding curve and the contents of the field memory on either side of the decoding curve, in the case of coincidence of the decoding curve with the inscribed curve;

FIG. 2 illustrates the detected signals on the decoding curve and the contents of the field memory on either side of the decoding curve, in the case of non-coincidence of the decoding curve with the inscribed curve;

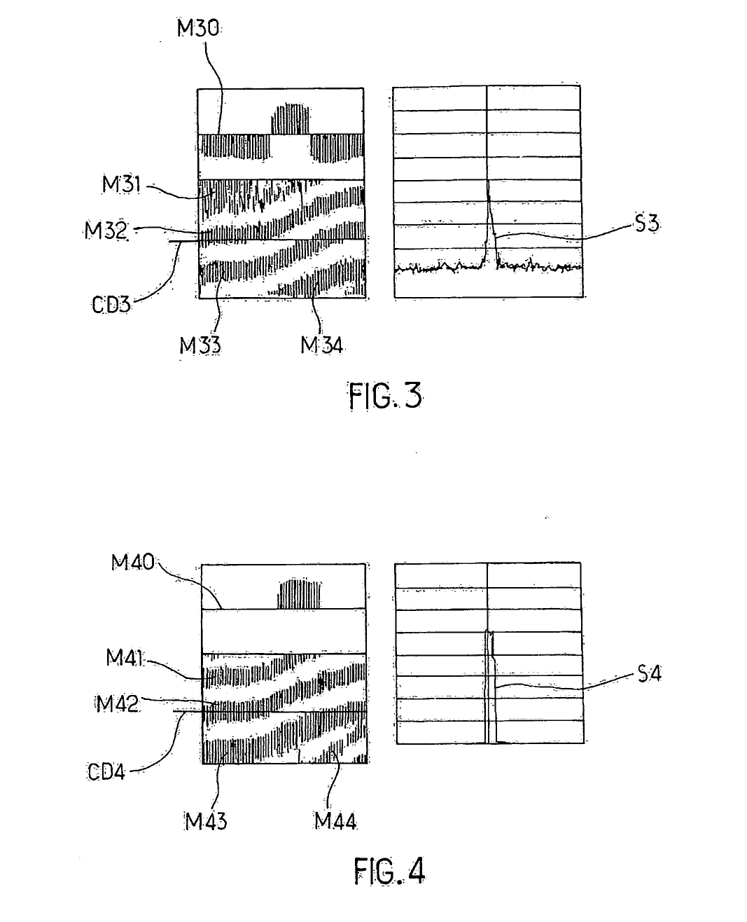

FIG. 3 illustrates the result of the decoding, the positive and negative signals detected on the decoding curve having been taken into account;

FIG. 4 illustrates the result of the decoding, the positive signals detected on the decoding curve having exclusively been taken into account;

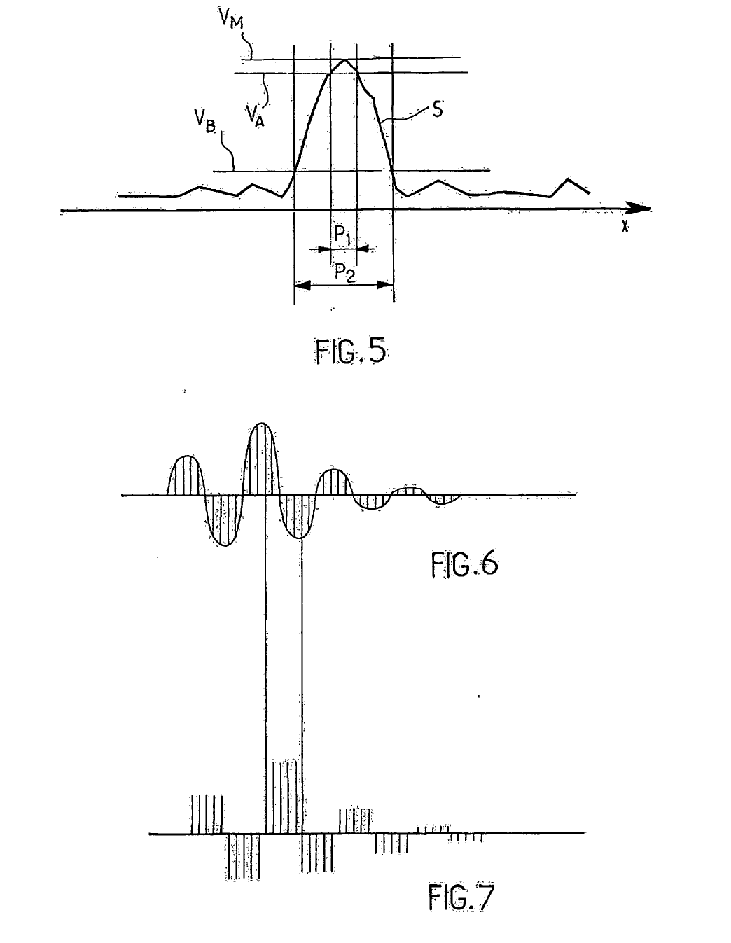

FIG. 5 illustrates a theoretical curve of the image of an obstacle in an analysis frame without any correction device;

FIG. 6 illustrates the envelope of a digitized signal versus time, read along a stored field line, before processing;

FIG. 7 illustrates the envelope of a digitized signal versus time, read along a stored field line, after processing;

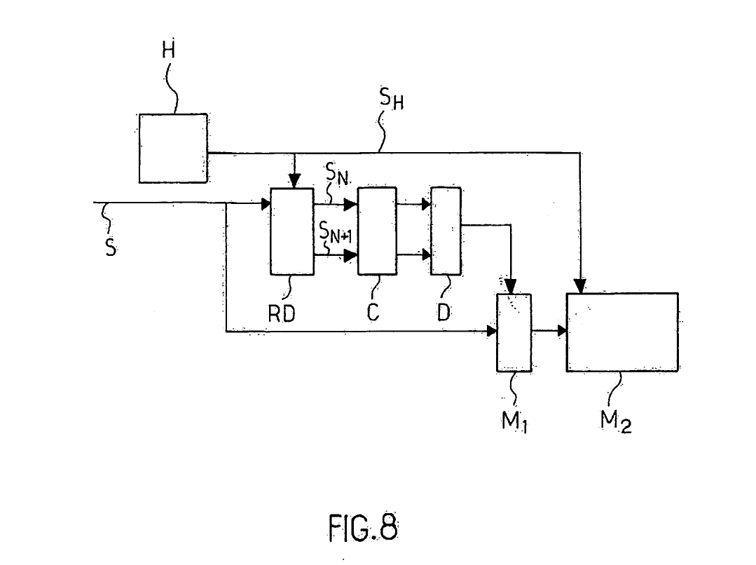

FIG. 8 illustrates a device for processing the digitized signal, read along a stored field line, the results of which are illustrated by FIGS. 6 and 7.

In a first example illustrated by FIGS. 1 to 4, the step for optimizing the spatial resolution, from information read in the field memory, comprises the evaluation of the horizontal difference between the decoding curve and the inscribed curve, and the correction of the amplitude of the measured signals according to this difference; thus, when the decoding curve does not exactly coincide with the inscribed curve, the decoding curve may successively detect positive and negative signals, the sum of said positive and negative signals tending towards 0.

Conversely, when the decoding curve exactly coincides with the inscribed curve, the decoding curve detects signals of the same polarity.

Thus, in the example illustrated in FIG. 1, the contents of the field memory are illustrated by the amplitude of the detected and stored signals M11, M12, M13, M14; the decoding curve CD1 almost exactly coincides with an inscribed curve illustrated by the detected and stored signals M13 in the field memory; thus, the amplitudes of the signals read in the field memory, illustrated by M10, are all of the same sign.

Conversely, in the example illustrated in FIG. 2, the contents of the field memory is illustrated by the amplitudes of the detected and stored signals M21, M22, M23, M24; the decoding curve CD2 does not coincide with any inscribed curve illustrated by the detected and stored signals in the field memory; the decoding curve is decentred and successively crosses several inscribed curves; thus, the amplitudes of the signals read in the field memory, illustrated by M20 are sometimes positive, sometimes negative.

Let S+ be the number of positive values, S− the number of negative values and SM the total number of positive and negative values on a decoding curve; therefore SM=(S+)+(S−).

With the assumption (theoretical case) that all the positive and negative values are of the same amplitude A, the detected amplitude after addition is: A0=SM×A.

In the case when the decoding curve does not coincide with the inscribed curve, the detected amplitude, after addition, becomes:

A0=A×(S+)−A×(S−); which may be written as:

A0=A×K, with K=SM−2×(S−).

The K factor depends on the difference between points of opposite signs; it will be equal to SM when all the values are of the same sign, and equal to zero when there are as many positive values as negative values.

It should be noted that the K factor is not constant because the inscribed values vary according to their horizontal position on the inscribed curve; indeed, the directivity of the reflecting obstacles causes a maximum of the detected signal in the line of sight of the obstacle, and therefore a weaker detected signal on the periphery around the line of sight.

Thus, the K factor should be replaced with a function f(k); this function may be determined theoretically or experimentally by moving the probe in front of a known obstacle and by measuring for each position of said probe, the amplitude of the reflected signal and the corresponding value of the K factor.

In the example illustrated in FIG. 3, the coincidence between the decoding curve CD3 and the inscribed curves M31, M32, M33, M34 is not achieved; indeed the signals M30 change sign when moving away from the centre of the decoding curve, and the number of signals of the same sign from the centre is all the smaller since the shift is large, so that the sum of the amplitudes decreases very rapidly.

The exact value of the amplitude A may then be simply obtained by dividing A0 by K. This method has the advantage of being able to be achieved very simply, as it is sufficient to perform the additions on the sign bit of the stored signal.

A very close method, based on the same principle, but the achievement of which is slightly more complex, may be used for carrying out this correction and also reducing the amplitude variations according to the position of the decoding curves relatively to the inscribed curves.

Indeed, if only the signals with the same sign around the central portion are added, and if this sum is divided by the number of signals with the same sign around this central portion, a very close value S3 is obtained, otherwise equal to the detected amplitude in the case of perfect coincidence between the decoding curve and the inscribed curve.

This process may be performed sequentially, by storing the sign of the signal at the centre of the decoding curve, and then by performing an analysis on either side of the centre, which analysis is stopped in the case of a detection of the change of sign of the detected signal; thus, the analysis consists of performing the sum of the detected signals, divided by the number of analyzed signals with the same sign; this value is stored for the relevant point; the operation is then performed in the same way for the other points to be analyzed, this processing may also be performed in parallel by logic circuits.

Moreover, this method excessively amplifies signals corresponding to noise or to signals reflected by obstacles located away from the point to be analyzed and the resulting image may well be confused.

In order to avoid such a drawback, it is considered that below a certain number of signals with the same sign, detected from the centre, this is no longer a defect, but background noise, and the sum of these signals is then no longer divided by their number, but by a constant value, generally the one corresponding to the total opening of the decoding curve.

In the example illustrated in FIG. 4, the coincidence between the decoding curve CD4 and the inscribed curves M41, M42, M43, M44, is not achieved; in the present case, only the central values with the same sign have been taken into account; the resulting curve S4 has an amplitude close to that of the resulting curve S3 and remains constant along a larger horizontal displacement on either side of the centre of the decoding curve. Thus, the accuracy on the amplitude of the detected signal is higher while adopting a larger step of analysis.

The different aforementioned processes contribute to optimizing the spatial resolution, from information read in the field memory; the possible association of processes allowing the processing rate of the signals in the analysis frame to be increased, contributes to improving the performances of the method described in Patents EP 0 825 453 B1 and EP 0 872 742 B1 filed on behalf of the applicant, notably in the analysis of parts with large dimensions at a high rate.

In this example, according to the invention, the step for optimizing the processing time, from information read in the analysis frame and during the storage phase in the field memory, first comprises the use of a variable horizontal step depending on the depth of analysis.

Indeed, the horizontal resolution is dependent on the depth of the obstacle; as an example, a depth resolution from 0.25 mm to 3 mm will become 1 mm to 30 mm in depth.

It is thus conceivable to adopt a variable horizontal step depending on the depth, said step being defined by the acoustic resolution at this depth; thus, by knowing the depth of the points to be analyzed, the analysis step will be stored beforehand and will allow a gain in time for analyzing the structure, close to 2-3.

The step for optimizing the processing time, from information read in the analysis frame and during the storage phase in the field memory, secondly comprises the change of the horizontal step in the vicinity of the obstacle.

Indeed, by detecting all the signals for which the amplitude is above the noise, with a large analysis step, it is possible to approximately localize said signals; a finer detection is then performed in the area containing each of said signals.

In the example illustrated in FIG. 5, a signal S illustrating a theoretical curve of the image of an obstacle in an analysis frame. Said signal S is characterized by a maximum amplitude V_{M} and a maximum amplitude noise level V_{B}, said theoretical curve being obtained with an analysis step close to zero.

Let V_{A} be the analysis level, less than the amplitude V_{M}, considered as the threshold for detecting signals to be analyzed; a detection error less than Δ=V_{M}−V_{A}, imposes an analysis step less than P₁, defined by the intersection of curve S and of analysis level V_{A}.

In the same way, in order to detect signals with an amplitude larger than the noise level, the analysis step will be substantially more significant, equivalent to P₂, defined by the intersection of curve S and of the maximum amplitude noise level V_{B}.

Thus, the detection of the signals will be carried out with an analysis step P₂, defined as the step corresponding to the detection threshold located above the noise level, allowing the search for signals with amplitude larger than the analysis level V_{A} to be carried out.

When an obstacle is detected at a certain abscissa xₚ, analysis of this obstacle consists of resuming the detection of said obstacle, from the abscissa xₚ−P₂ with an analysis step P₁. Said analysis is completed in the vicinity of the obstacle, when the detected signal is less than the noise level V_{B} and the abscissa larger than xₚ.

It is therefore conceivable to adopt a variable horizontal step depending on the encountered obstacles, said step being relatively large, allowing high rates; thus, having detected an obstacle to be analyzed, the analysis step will be reduced in the detection area of said obstacle; the processing time is practically not affected by the reduced step analysis as regards the gain in time provided by a high initial step.

The step for optimizing the processing time, from information read in the analysis frame and during the storage phase in the field memory, thirdly comprises processing of the detected signals.

Indeed, signals reflected or transmitted by an obstacle, as sampled signals, are stored in each field line of the field memory; thus, the amplitude of the samples represents the envelope of said detected signals; the sampling frequency is larger than the frequency of the detected signals in order to detect the extrema of said signals.

In the example illustrated in FIG. 6, the sampling period of the detected signal is about ten times smaller than the period of said detected signal.

The processing of the sampled signal consists of detecting the extrema of said sampled signal, storing the samples with a corresponding amplitude during a half period of the detected signal, and storing in the field line of the field memory, not the totality of the samples of said sampled signal, but only the amplitude samples corresponding to the extrema.

In the example illustrated in FIG. 7, the sampled signal consists of extrema of the original signal, illustrated in FIG. 6; thus, the analysis step of the processed signal, may attain the half period of the original signal, while retaining the required accuracy on the amplitude of the detected signals.

The aforementioned processing may be carried out in software or in hardware; in the example illustrated in FIG. 8, the processing is carried out by hardware means.

A clock unit H delivers a clock signal S_{H} which is applied to a shift register unit (RD) with two stages on the one hand, and to a main memory unit M₂ on the other hand; the original signal S, sampled at the frequency of said clock signal S_{H}, is applied to the input of the shift register unit (RD) on the one hand, and to the input of the buffer memory M₁ on the other hand.

The shift register unit (RD) delivers signals S_{N} and S_{N+1} corresponding to two successive samples of said signal S; the aforesaid signals S_{N} and S_{N+1} are applied to both inputs of a comparator C, the two outputs of which switch from state 1 to state 0 according to whether sample N is larger or smaller than sample N+1; the transition from 1 to 0 corresponds to an extremum of the signal S.

Said transition controls a detection circuit D which controls the storage of the signal S in said buffer memory M₁. The output of memory M₁ is then applied to the input of the main memory M₂.

Thus, the main memory M₂ contains the values of the extrema at each half period of the original signal S.

The whole of the aforementioned processes for optimizing spatial resolution, from information read in the field memory, and for optimizing the processing time of the detected signals may be applied separately or in combination; they thereby contribute in allowing the processing rate of the detected signals to be increased while retaining very high spatial resolution, and allowing three-dimensional structures to be analyzed three-dimensionally at a high rate.

1. A method for scanning and analyzing a three-dimensional structure by suitable processing signals representing of waves, notably ultrasonic waves, reflected or transmitted by said three-dimensional structure, said processing consisting of reconstructing or analyzing the three-dimensional structure from information read in a field memory, wherein for each structure point, the positions of the field memory containing the signals detected by the detection elements, corresponding to the waves reflected or transmitted by this point, are calculated, which information in the field memory is read by a decoding curve,
said method detecting the sign of the detected signals, detecting a useful area of said decoding curve wherein the signals are of the same sign and calculating the position of the analyzed point from integration of the amplitudes of the detected signals in said useful area of said decoding curve.

2. The method according to claim 1, implementing
detection of positive and negative signals,
detection of the centre of said decoding curve,
storing the sign of said signals at the centre of the decoding curve,
detection of a useful area of the decoding curve wherein the signals are of the same sign,
adding said signals of the same sign in the useful area of the decoding curve,
dividing said sum by the number of analyzed signals of the same sign or by a predetermined constant value corresponding to the total opening of the decoding curve,
storing the result obtained for the analyzed point of the three-dimensional structure.

3. The method according to claim 1, comprising, from information read in an analysis frame and during the storage phase in the field memory:
using a variable horizontal step depending on the depth, and/or
changing the horizontal step in the vicinity of the detected obstacle, and/or
processing the signals detected, prior to the storage phase.

4. The method according to claim 3, using a variable horizontal step depending on the depth comprises measuring the spatial resolution versus the depth and storing said resolution.

5. The method according to claim 3, changing the horizontal step in the vicinity of the obstacle comprises detection of all signals for which the amplitude is above the noise, localization of said detected signals, and finer detection in the area containing each of said detected signals.

6. The method according to claim 3,
wherein the processing of the signals detected prior to the storage phase comprises detection of the extrema of said sampled signals, storage of samples with a corresponding amplitude during a half period of said detected signals, and storage in the field line of the field memory of samples with an amplitude corresponding to the extrema.
